(19) United States
(12) Patent Application Publication
(10) Pub. No.: US 2017/0209056 A1
(43) Pub. Date: Jul. 27, 2017

(54) IMPLANTABLE SELF-CALIBRATING SENSOR ASSEMBLIES AND ASSOCIATED METHODS

(71) Applicants: UNIVERSITY OF WASHINGTON, Seattle, WA (US); AQUEDUCT CRITICAL CARE, INC., C/O MERCURY MEDTECH AND LAKE WASHINGTON INSTITUTE OF TECHNOLOGY, Bothell, WA (US)

(72) Inventors: Samuel R. Browd, Seattle, WA (US); Barry Lutz, Seattle, WA (US); Thomas J. Clement, Bothell, WA (US); Joel Relethford, Bothell, WA (US); Jared Salstrom, Bothell, WA (US)

(21) Appl. No.: 15/128,218

(22) PCT Filed: Apr. 7, 2015
(86) PCT No.: PCT/US15/24762

§ 371 Date: Sep. 22, 2016

Related U.S. Application Data
(60) Provisional application No. 61/976,200, filed on Apr. 7, 2014.

(51) Int. Cl. A61B 5/03; A61M 27/00; A61B 5/00

(52) U.S. Cl. CPC A61B 5/032; A61B 5/6847; A61M 27/006; A61B 2560/0223; A61M 2230/005; A61M 2205/3331

(57) ABSTRACT

Self-calibrating sensor assemblies for excess body fluid drainage systems are disclosed herein. Self-calibrating sensor assemblies can include a sensor assembly engaged with a flexible interface member of a drainage catheter. The assembly includes a sensor having a body and a shaft extending from the body. A contact member is slidably mated with the shaft and coupled to the flexible interface member. A resilient member is coupled to the sensor shaft and disposed between the contact member and the body. An actuator moves the sensor between a first position and a second position with respect to the drainage catheter. In the first position the sensor is positioned to measure the pressure and/or force at the flexible interface member, and in the second position the resilient member exerts a known force on the sensor.

### CROSS-REFERENCE TO RELATED APPLICATION(S)

This application claims the benefit of pending U.S. Provisional Patent Application No. 61/976,200, filed Apr. 7, 2014, which is incorporated herein by reference in its entirety.

### TECHNICAL FIELD

The present technology relates generally to draining excess body fluids. In particular, several embodiments are directed toward implantable self-calibrating sensor assemblies for body fluid drainage systems and associated methods.

### BACKGROUND

A variety of medical conditions cause a collection of excess body fluids within the human body. Hydrocephalus, for example, is an accumulation of excess cerebrospinal fluid (“CSF”) in the ventricles of the brain that increases intracranial pressure (“ICP”). This condition can be caused by the inability to reabsorb CSF, impaired CSF flow, or excessive production of CSF. Acute accumulations of excess CSF can also occur from brain trauma, brain hemorrhaging, strokes, brain tumors, spinal fluid leaks, meningitis, and brain abscesses. When left untreated, hydrocephalus and other excess accumulations of CSF can progressively enlarge the ventricles of the brain, which increases ICP. When left untreated, high ICP results in convulsions, mental disabilities, and eventually death.

Treatment for hydrocephalus generally requires the installation of a CSF shunt that drains CSF from the brain to an alternate location that can collect the excess CSF or reabsorb it into the body. A ventriculoperitoneal shunt (“VPS”), for example, includes a subcutaneously installed catheter inserted in the lateral ventricle (i.e., a site of excess CSF) and in fluid communication with the peritoneal cavity to facilitate reabsorbtion of the excess CSF into the body. A mechanical valve, generally implanted flush with the skull, can regulate CSF flow through the catheter.

Similar to hydrocephalus, acute accumulations of CSF are treated by shunting excess CSF to an alternate location. For example, temporary CSF diversion generally includes the installation of an external ventricular drain (“EVD”) that funnels CSF from the lateral ventricle to an external drainage chamber, thereby reducing the intracranial CSF volume and lowering ICP. Alternatively, temporary CSF diversion can include placing a lumbar drain (“LD”) at the base of the spine, and draining CSF from the lumbar region to an external drainage chamber. Despite having different insertion points, EVDs and LDs use the similar components to control drainage.

In general, temporary and more permanent CSF diversion devices (e.g., VPSs) include similar features, and are therefore subject to many of the same technical challenges and complications. For example, it is important to accurately measure a patient's ICP to ensure that the flow rate through the shunt provides the necessary pressure relief to the brain. In addition, accurate ICP measurements are helpful in determining whether the CSF diversion device is functioning properly. The inlet of the catheter, for example, can incur in-growth of intraventricular tissue. Valves can fail due to debris build-up (e.g., blood, protein) within the valve, and the outlet of the catheter can fail by fracturing, becoming obstructed, or tethering within scar tissue. Moreover, infection can be a significant risk factor both during and after implantation of a CSF shunt. When an infection occurs, the entire CSF shunt must be removed, and the patient must generally undergo 10-14 days of IV antibiotics and re-internalization of a new CSF shunt. These mechanical failures, infections, and other complications cause a majority of implanted CSF shunts to fail within two years and nearly all shunts fail within ten years. Due to this unreliability and the necessity to locally monitor and adjust ICPs, conventional CSF shunts require frequent monitoring and intervention by medical professionals.

### BRIEF DESCRIPTION OF THE DRAWINGS

FIG. 1A is a schematic view of an internal body fluid drainage system installed within a patient in accordance with an embodiment of the present technology.

FIG. 1B is a schematic view of an external body fluid drainage system installed in a patient in accordance with an embodiment of the present technology

FIG. 2A is an exploded schematic view of components of a self-calibrating sensor assembly configured in accordance with an embodiment of the present technology.

FIG. 2B is a schematic view of a self-calibrating sensor assembly engaged with a drainage catheter in a sensing mode configured in accordance with an embodiment of the present technology.

FIG. 2C is a schematic view of the self-calibrating sensor assembly of FIG. 2B in a calibrating mode.

FIG. 3A is a schematic view of a self-calibrating sensor assembly engaged with a drainage catheter in a sensing mode configured in accordance with another embodiment of the present technology.

FIG. 3B is a schematic view of the self-calibrating sensor assembly of FIG. 3A in a calibrating mode.

FIGS. 4A and 4B are schematic views of self-calibrating sensor assembly components in accordance with embodiments of the present technology.

FIG. 5A is a schematic view of components of a self-calibrating sensor assembly in a sensing mode configured in accordance with an embodiment of the present technology.

FIG. 5B is a schematic view of the components of the self-calibrating sensor assembly of FIG. 5A in a calibration mode.

FIG. 6A is an exploded schematic view of components of a self-calibrating sensor assembly configured in accordance with another embodiment of the present technology.

FIG. 6B is a schematic view of a self-calibrating sensor assembly engaged with a drainage catheter in a sensing mode configured in accordance with another embodiment of the present technology.

FIG. 6C is a schematic view of the self-calibrating sensor assembly of FIG. 6B in a calibrating mode.

FIG. 7A is a graph showing an example of a single-point calibration.

FIG. 7B is a graph showing an example of a two-point calibration.

FIG. 7C is a graph showing an example of a three-point calibration.

### DETAILED DESCRIPTION

The present technology is directed to devices, systems, and methods for draining excess body fluids and self-calibrating sensor assemblies configured to determine pressure at the site of excess body fluid. In one embodiment, for example, a body fluid drainage system can be installed between a site of excess body fluid in a patient, such as within a patient's head, and a second location (e.g., an external receptacle, an internal cavity) that can collect and/or reabsorb the excess body fluid. The body fluid drainage system also includes a self-calibrating sensor assembly for determining pressure within a drainage catheter.

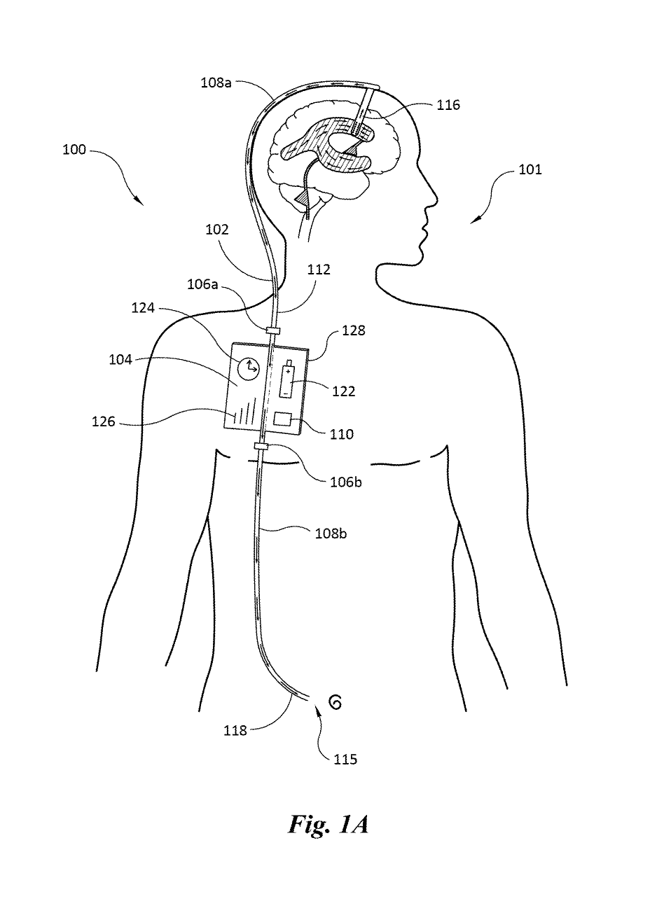

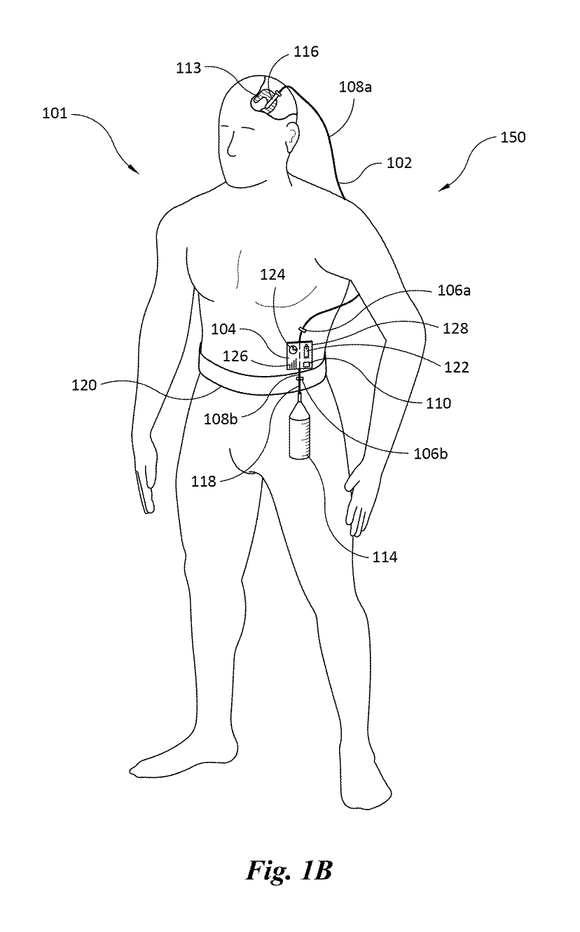

Certain specific details are set forth in the following description and in FIGS. 1A-7C to provide a thorough understanding of various embodiments of the technology. For example, several embodiments of self-calibrating sensor assemblies for body fluid drainage systems are described in detail below. The present technology, however, may be used in a wide variety of applications, and need not be limited to measuring pressure of excess body fluids. Additionally, the term “catheter” is used broadly throughout the application to refer to any suitable tubing or structure that includes a lumen through which fluids can flow. Other details describing well-known structures and systems often associated with CSF and other body fluid drainage systems, shunts, biomedical diagnostics, etc. have not been set forth in the following disclosure to avoid unnecessarily obscuring the description of the various embodiments of the technology. A person of ordinary skill in the art, therefore, will accordingly understand that the technology may have other embodiments with additional elements, or the technology may have other embodiments without several of the features shown and described below with reference to FIGS. 1A-7C.

### Selected Embodiments of Body Fluid Drainage Systems

FIG. 1A is a schematic view of an internal body fluid drainage system 100 (“drainage system 100”) implanted in a human patient 101 in accordance with an embodiment of the present technology. The drainage system 100 can include a catheter 102, a valve device 104 over an exterior surface 112 of the catheter 102, and one or more sensor assemblies 106 (identified individually as a first sensor assembly 106a and a second sensor assembly 106b). The drainage system 100 can also include a controller 110 operatively coupled to the valve device 104 and/or the sensor assemblies 106. As described in further detail below, the valve device 104 can apply incremental forces to the exterior surface 112 of the catheter 102 to regulate body fluid flow through the catheter 102, and the controller 110 can alter the level of force applied by the valve device 104 on the catheter 102 in response to measurements (e.g., pressure, flow rate) taken from the sensor assemblies 106. As described in more detail below, in some embodiments the sensor assemblies 106 can be self-calibrating assemblies configured to periodically re-calibrate via application of a known force or forces. The controller 110 can be in communication with the sensor assemblies 106 in order to coordinate calibration processes for the sensor assemblies 106 with normal diagnostic operation of the sensor assemblies 106. For example, during calibration of the sensor assemblies 106, the controller 110 may maintain the valve device 104 at a fixed position without regard to output of the sensor assemblies 106. Once calibration has been completed, the controller 110 can continue normal control of the valve device 104 based on output from the sensor assemblies 106.

As shown in FIG. 1A, the catheter 102 can include a proximal portion 108a and a distal portion 108b opposite the proximal portion 108a. The proximal and distal portions 108a-b of the catheter 102 can be an integrally formed tube or include two or more separate tubes joined together using suitable fastening methods (e.g., gluing) known in the art. The catheter 102 can be made from a range of polymers, such as silicone, latex, thermoplastic elastomers, and/or other suitable tubing materials. In selected embodiments, portions of the catheter proximate to the valve device 104 can include compressible peristaltic pump tubing (e.g., silicone rubber, polyvinyl chloride), reduced fouling surfaces, tubing with different mechanical compliances, and/or other durable elastomeric materials that resist fatigue. In other embodiments, the catheter 102 can be made from tubing with biocides and/or other anti-biofouling agents that prevent organisms from entering the drainage system 100 and causing infection. When the catheter 102 includes different materials and/or sections of tubing, the different materials and/or portions can be sealed together with adhesives and/or other fasteners that provide a liquid-tight seal.

The proximal portion 108a of the catheter 102 is adapted to be positioned at a site of excess body fluid and the distal portion 108b can be placed in fluid communication with an internal receptacle that collects and/or absorbs the body fluid. The proximal portion 108a of the catheter 102 can include an inlet region 116 with one or more openings (not visible) in fluid communication with the site of excess body fluid such that the body fluid can flow into the catheter 102. In the embodiment illustrated in FIG. 1A, for example, the inlet region 116 of the catheter 102 is installed (e.g., via a burr hole) into a ventricle 113 of the patient's brain to receive excess CSF. After entering the drainage system 100, the body fluid can travel in an antegrade flow through the catheter 102 to the distal portion 108b. The distal portion 108b can include an outlet region 118 that expels the excess body fluid into an internal location. For example, the outlet region 118 can be placed in fluid communication with the patient's peritoneal cavity 115, where excess body fluid can reabsorb into the body. In other embodiments, the outlet region 118 can expel the body fluid into the atrium of the heart, the pleural lining of the lung, the gallbladder, and/or other suitable terminal locations.

The valve device 104 can be positioned between the proximal and distal portions 108a-b of the catheter 102 to regulate the body fluid flow through the drainage system 100. As shown in FIG. 1A, for example, the valve device 104 can be implanted in a subclavicular pocket of the patient 101. In other embodiments, however, the valve device 104 can be installed in a prefascial or subfascial intra-abdominal region. This intra-abdominal positioning is particularly suited for neonates to ease exchange of the valve device 104 as the child grows, but also facilitates accessibility to the valve device 104 for adults. Advantageously, placement of the valve device 104 in either the subclavicular pocket or the intra-abdominal region negates the need to shave the patient's scalp to perform cranial surgery in the event that a component requires replacement or repair, and thus avoids the need for repeated incisions in the scalp that can cause devascularization, poor wound healing, and/or infection. The intra-abdominal valve device 104 also eases the periodic replacement of batteries or other power sources. In other embodiments, the valve device 104 can be installed subcutaneously in other regions of the torso or between another site of excess body fluid and a receptacle that can collect and/or reabsorb the body fluid. In further embodiments, the valve device 104 can be miniaturized such that it can be implanted under the scalp.

The sensor assemblies 106 are configured to measure pressure within the catheter 102, flow rate of the body fluid through the catheter 102, and/or other desired measurements associated with body fluid drainage through the drainage system 100. Pressure sensors can be small electrical sensors positioned along the drainage device 100. In some embodiments, the sensor assemblies 106 can additionally measure flow rate of body fluid through the catheter, for example with a non-electrical Rotameter that uses a local or remote sensor to read the position of a weighted or buoyant ball that rises and falls within the catheter 102 in proportion to the flow rate. In other embodiments, the body fluid flow rate can be measured using what is known in the art as the “ice cube test.” An improved version of such a flow rate sensor includes a resistive electrical heater and temperature sensor embedded in the body fluid flow, rather than an external heater/cooler and an external temperature measurement device used in conventional ice cube tests. In further embodiments, body fluid flow rate can be measured using what is known as a “tick-tock chamber” that senses the rate that specialized chambers refill with the body fluid within the catheter 102.

As shown in FIG. 1A, the sensor assemblies 106 can be positioned proximate to the outlet and inlet to the valve device 104. Accordingly, the first sensor assembly 106a can measure the flow rate and/or the pressure within the proximal portion 108a before it enters the valve device 104 and the second sensor assembly 106b can measure the flow rate and/or pressure within the distal portion 108b as it exits the valve device 104. This information can be used to ensure the valve device 104 generates the desired drainage rate, to monitor patient orientation, to perform diagnostics on the drainage system, and/or derive other desired measurements or characteristics. In other embodiments, the drainage system 100 can include more or less sensor assemblies 106. For example, a pressure sensor assembly 106 can be positioned proximate to the inlet region 116 to measure ICP directly.

The sensor assemblies 106 can also be used to derive a pressure at a desired location (e.g., the Foramen of Monroe for ICP) spaced apart from the sensor assemblies 106. For example, the sensor assemblies 106 that are positioned proximate to the valve device 104 in the torso of the patient 101 can be used to derive ICP. As shown in FIG. 1A, the sensor assemblies 106 can be positioned on either side of the valve device 104 to measure pressure upstream and downstream of the valve device 104. When the patient 101 is upright (i.e., standing), the first sensor assembly 106a at the proximal portion 108a can measure a pressure that is substantially equal to the ICP plus the pressure head created by the body fluid in the proximal portion 108a above the first sensor assembly 106a. The second sensor assembly 106b at the distal portion 108b can measure a pressure substantially equal to the pressure at the outlet region 118 (e.g., the peritoneal cavity 115; as is known in the art, the pressure is approximated as zero relative to atmosphere) plus the negative pressure created by the body fluid in the distal portion 108b below the second sensor assembly 106b. The pressures from the upstream and downstream sensor assemblies 106 can be combined to derive the true ICP. For example, when the valve device 104 is positioned midway between the ventricle 113 and the outlet region 118, the summation of the two pressure measurements from the sensor assemblies 106 negates the contribution of pressure head and provides the true ICP.

In other embodiments, a pressure reference line can be coupled to the drainage system 100 and used to compensate for changes in patient position. The pressure reference line measures the pressure head between a desired reference location and the sensor assembly 106 at the valve device 104 directly. As such, the desired pressure measurement (e.g., ICP) is simply the difference between the two measured pressures as taken from two independent sensors (i.e., the pressure reference line sensor and the drainage line sensor) or a single differential pressure sensor.

The drainage system 100 can also include an orientation sensor (not shown) to accurately measure a desired pressure (e.g., ICP) regardless of the orientation of the patient 101. For example, the orientation sensor can include an accelerometer, inclinometer, and/or other orientation sensing device. The orientation sensor is used to determine the angle of repose (i.e., standing, lying, or therebetween); such that the measured angle and the known length of the proximal portion 108a of the catheter 102 can be used to calculate the pressure head. The pressure head can be subtracted from the measured pressure to calculate the true ICP.

The controller 110 (e.g., a microprocessor) is configured to read the measurements taken from the sensor assemblies 106 (e.g., pressure, flow rate, orientation, etc.), store such measurements and other information in a database, adjust the position of the valve device 104, and/or carry out algorithms to regulate fluid flow through the drainage device 100. For example, the controller 110 can compare pressure measurements from the sensor assemblies 106 with a desired ICP to determine whether to incrementally open or close the valve device 104 and by what percentage. When the pressure is lower than a desired pressure, for example, the controller 110 can incrementally close the valve device 104 to increase the resistance to antegrade flow through the catheter 102. If the sensed pressure is higher than desired, the controller 110 can incrementally open the valve device 104 to decrease the resistance to antegrade flow. Similarly, the controller can also compare the sensed flow rate with a desired flow rate and adjust the position of the valve device 104 accordingly. The controller 110 can also carry out an algorithm that moves the valve device 104 a predetermined amount each time a measurement outside of a desired limit (e.g., desired CSF range) is detected. Such a control algorithm can also relate the incremental movement of the valve device 104 to the magnitude of the difference between a desired and a measured value. In other embodiments, a proportional-integral-derivative (“PID”) control algorithm or variations thereof (e.g., P-only, PI-only) can control the movement of the valve device 104. As such, the controller 110 can manage body fluid flow in real-time to maintain the ICP and/or other desired parameter within appropriate limits across a range of changes in pressure or body fluid generation rate caused by physiologic processes (e.g., valsalva maneuvers, changes in body orientation).

The controller 110 can include algorithms that save power. For example, a tolerance window on the control parameter (e.g., ICP or CSF flow rate) can be defined such that the valve device 104 does not change position within the tolerance window. As another example, the time between sensor measurements can be adjusted based on the error between the desired set point and the measured value, such that less frequent measurements are made during periods of small error. These power-saving control algorithms can also be adapted to the dynamics of the specific application. During CSF drainage, for example, significant changes in CSF production may occur over several hours such that only infrequent sensor measurements and valve device 104 movements are necessary for adequate flow control. As such, the controller 110 can be configured to ignore unimportant transient conditions (e.g., ICP oscillations due to the cardiac cycle, ICP increases due to coughing or movement) removed by averaging sensor measurements and/or frequency filtering.

Additionally, the controller 110 can include logic to clear the valve device 104 of obstructions by incrementally opening the valve device 104 until the obstruction clears. For example, the controller 110 can be configured to maintain a desired ICP such that when an obstruction within the valve device 104 causes an increase in the measured pressure, the control algorithm (e.g., a proportional-integral-derivative) incrementally or fully opens the valve device 104 to decrease the resistance to antegrade flow. This incremental opening of the valve device 104 allows the obstruction to flow through the valve device 104 such that the drainage system 100 can maintain the desired ICP. As described in further detail below, in other embodiments, the controller 110 can include logic that clears and/or prevents obstructions by flushing the catheter 102 with body fluid.

As further shown in FIG. 1A, the drainage system 100 can include a time keeping device 124 (e.g., clock, timer, etc.) that is operatively coupled to the controller 110. The controller 110 can use the time keeping device 124 to sense pressure and/or flow rate at preset time intervals (e.g., once a minute). Additionally, the controller 110 can use the time keeping device 124 to periodically flush the catheter 102 and/or periodically run diagnostics.

Additionally, as shown in FIG. 1A, the drainage system 100 can also include a power source 122 for the valve device 104 and/or other electrical features (e.g., the time keeping device 124, the sensor assemblies 106, etc.). The power source 122 can be stored locally within the drainage system 100. As such, the power source 122 can thus include a lithium-ion cell, a rechargeable battery, and/or other suitable portable power sources. In selected embodiments, the internally installed power source 122 can be recharged remotely using inductive coupling, kinetic energy generation by M2E of Boise, Id., and/or other remote recharging methods known in the art. In other embodiments, the drainage system 100 can connect to an external recharging station.

In selected embodiments, the controller 110 can be operatively coupled to a wireless communication link 126, such as a WiFi connection, radio signal, and/or other suitable communication links that can send and/or receive information. The wireless communication link 126 allows measurements from the sensor assemblies 106 and/or other information to be monitored and/or analyzed remotely. For example, the wireless communication link 126 allows measurements recorded from the sensor assemblies 106 to be accessed at a doctor's office, at home by the patient 101, and/or at other remote locations. Additionally, the drainage system 100 can use the wireless communication link 126 to receive information at a WiFi hot spot or other remotely accessible locations. This allows a remote physician to inquiry the drainage system 100 regarding particular measurements (e.g., ICP), instruct the controller 110 to adjust the valve device 104 accordingly, and/or program sophisticated algorithms onto the controller 110 for the drainage system 100 to carry out. Accordingly, the drainage system 100 can provide more expedient, sophisticated, and personalized treatment than conventional CSF shunts, without requiring frequent in-office visits.

As further shown in FIG. 1A, the valve device 104, the controller 110, and/or other subcutaneously implanted features of the drainage system 100 can be enclosed within a housing 128. Accordingly, the housing 128 can be made from a biocompatible material that protects the devices stored within from tissue ingrowth, body fluids, and/or other internal bodily features that may interfere with the operability of the drainage system 100. In selected embodiments, the housing 128 can also form a magnetic shield over the devices within it such that the patient 101 can undergo magnetic resonance imaging (“MRI”) and similar procedures without removing the drainage system 100.

In operation, the drainage system 100 can have generally low power consumption. For example, the drainage system 100 requires minimal, if any, continuous power. In one embodiment, the time keeping device 124 is the only feature of the drainage system 100 that continuously draws from the power source 122. Other devices can draw from the power source 122 intermittently as needed. For example, the sensor assemblies 106 and/or other sensing devices can sense pressure at preset intervals (e.g., once per minute) and only draw from the power source 122 at that time. Similarly, any diagnostics and/or forced flows (e.g., backflushing, described below) only occur periodically and thus only require power occasionally. In selected embodiments, the valve device 104 only requires power when it changes position to adjust the pressure and/or flow rates. Without the need for any continuous substantial power, the drainage system 100 consumes much less power than would be required using a pump to drive body fluid. As described below, the drainage system 100 can also include a hybrid mechanical and electrical device that reduces the required frequency of actuator movements, and thus further reduces power consumption. Accordingly, the drainage system 100 can be configured such that the power source 122 runs the drainage system 100 for extended periods of time (e.g., five or more years), and therefore does not necessitate frequent surgeries to replace the power source 122.

Optionally, the drainage system 100 can also include a pump (e.g., an electro-osmotic pump) that can be activated to drive body fluid flow through the drainage system 100. For example, the controller 110 can include logic that activates the pump when the orientation of the patient 101 is such that the body fluid flows in the reverse direction (i.e., retrograde flow) through the catheter 102. In other embodiments, the drainage system 100 can include other suitable devices and features that facilitate the controlled drainage of body fluids.

The subcutaneously installed drainage system 100 shown in FIG. 1A can also include features that limit the risk of infection during and after implantation. For example, components of the drainage system 100 (e.g., the catheter 102, the housing 128) can include anti-fouling coatings and/or antibiotic impregnated materials. In selected embodiments, short-term thermal cooling and heating can be applied to the drainage system 100 as a whole or components thereof to reduce bacterial colonization during the perioperative period. In other embodiments, the housing 128, the valve device 104, and/or other portions of the drainage system 100 can be magnetized or otherwise treated to reduce bacterial growth and contamination.

FIG. 1B is a schematic view of an external body fluid drainage system 150 (“drainage system 150”) implanted in the patient 101 in accordance with an embodiment of the present technology. The drainage system 150 includes features generally similar to the drainage system 100 described above with reference to FIG. 1A. For example, the drainage system 150 can include the catheter 102 having the proximal portion 108a and the distal portion 108b, the valve device 104 positioned therebetween, the sensor assemblies 106, and the controller 110 operatively coupled to the sensor assemblies 106 and the valve device 104. Additionally, like the internal drainage system 100 described above, the external drainage system 150 can regulate CSF or other excess body fluid flow using sophisticated and individualized methods, and do so while operating as a low power system. However, the drainage system 150 shown in FIG. 1B is installed externally, between the ventricle 113 and an external receptacle 114. The external receptacle 114 can be placed in fluid communication with the outlet region 118 of the catheter 102 such that it can collect the excess body fluid. As such, the external receptacle 114 can be a bag or container made from a range of polymers (e.g., silicone, polyvinyl chloride) and/or other suitable materials for storing body fluids.

In the illustrated embodiment, the external receptacle 114 is secured to the midsection of the patient 101 with a belt 120 such that the patient 101 can remain mobile as the drainage system 150 removes the excess body fluid. As shown in FIG. 1B, the belt 120 can also carry the housing 128 that contains the valve device 104, the controller 110, and/or other devices that operate the drainage system 150. The externally positioned housing 128 can be made from a durable material (e.g., plastic) that can withstand the rigors of the outside environment and substantially protect the components within. Snaps, thread, hooks, and/or other suitable fasteners can be used to secure the external receptacle 114 and/or the housing 128 to the belt 120. In other embodiments, the external receptacle 114 and/or the housing 128 can be secured to other portions of the patient 101 that do not substantially inhibit the patient's mobility.

In further embodiments, such as when the drainage system 100 is used for temporary shunting of acute accumulation of the body fluid, the external receptacle 114 can be hung on a pole commonly used for IV bags or otherwise affixed to an external structure. Additionally, for temporary drainage, the devices within the housing 128 can also be positioned apart from the patient 101, such as on a console connected with a power source.

### Selected Embodiments of Self-Calibrating Sensor Assemblies

Implantable sensor assemblies are important diagnostic and interventional devices used for measuring physiological parameters that are difficult to measure noninvasively. However, implantable sensor assemblies present certain problems. For example, such assemblies should be bio-compatible, MRI-safe (i.e., the presence of the sensor when used during MRI presents no additional risk to the patient), and/or MRI-compatible (i.e., the presence of the sensor is MRI-safe and will not significantly affect the quality of the diagnostic information, nor will its operation be significantly affected by the MRI). Size and power constraints are also especially pronounced for implantable sensors, which are typically intended for long-term use. Long-term pressure measurement using implantable sensor assemblies can be difficult due to sensor drift. While sensor re-calibration can correct for sensor drift, typical calibration techniques are not possible when the sensor is implanted in the body.

Embodiments of the present technology allow for sensor calibration at desired intervals in an implanted or external device. The sensor assembly can allow for temporary application of a known force (e.g., the sensor can be advanced against a spring causing it to compress to a known tension) thereby providing at least one calibration reference point. In some embodiments, calibrating sensor-assemblies as disclosed herein can be used in conjunction with the body fluid drainage system described above with respect to FIGS. 1A and 1B. Although the technology disclosed herein is described in certain examples in relation to implantable sensors, it may also be applied to sensors external to the body (e.g., the sensor assemblies 106 of FIG. 1B). The term “force” is used broadly herein, and in some embodiments “pressure” is an equally valid term. Additionally, in selected embodiments, a sensor assembly can measure force or pressure of the catheter.

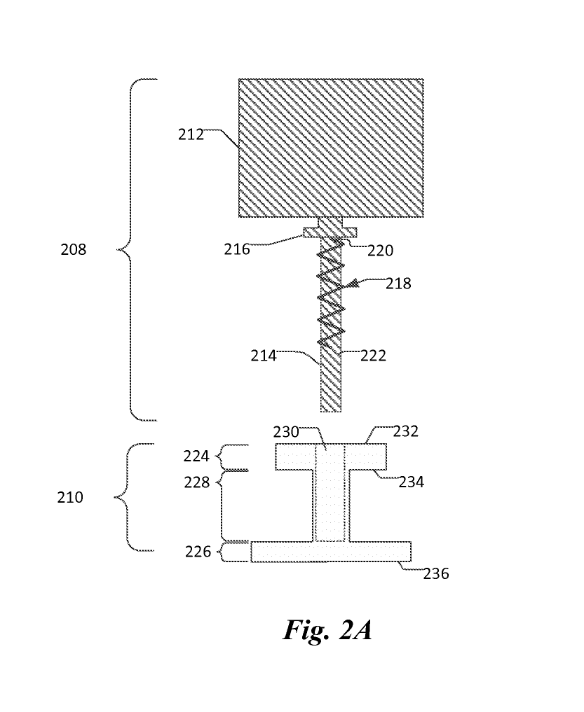

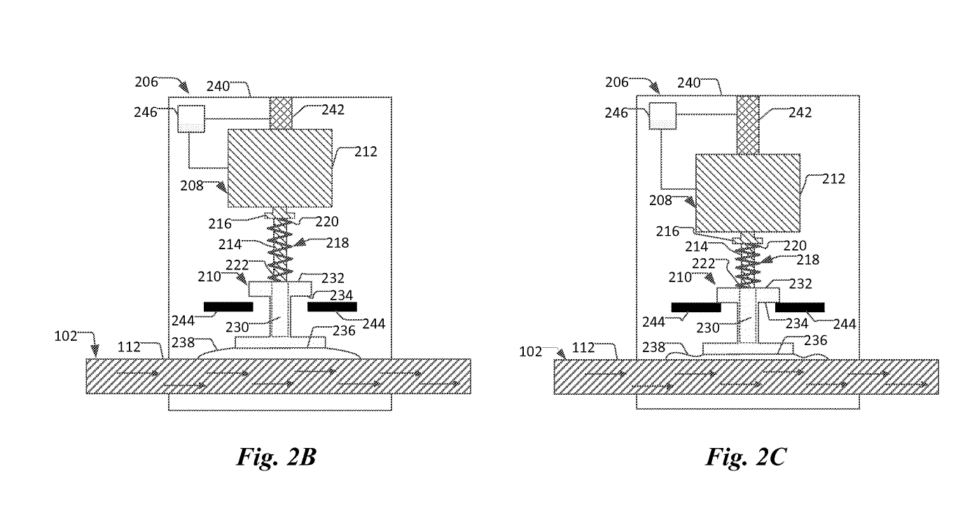

FIG. 2A is an exploded schematic view of certain components of a self- calibrating sensor assembly 206. The sensor assembly 206 includes, for example, a sensor 208 and a contact member 210 configured to slidably mate with the sensor 208. The sensor 208 includes a body 212 at the proximal portion. A shaft 214 projects distally from the body 212. A collar 216 connects the body 212 and the shaft 214. The collar 216 can have a width greater than the width of the shaft 214. In some embodiments, the collar 216 can be integrally formed with the shaft 214, while in other embodiments the collar 216 can be a separate component fixedly attached to the shaft 214. The sensor 208 can be configured to measure the force applied to the shaft 214 and/or the force applied to the collar 216. The sensor 208 can be, for example, a transducer configured to generate an electrical signal indicative of the force being measured. Examples of such transducers include, for example, piezoelectric, capacitive, strain gauge-based sensors, or other suitable type of sensors for measuring the force applied to the shaft 214 and/or the collar 216.

A resilient member 218 is coupled to the shaft 214. For example, the resilient member 218 can be a helical spring wound around the shaft 214. The resilient member 218 has a proximal end 220 that is coupled to the collar 216 and a distal end 222 opposite the proximal end 220. The resilient member 218 is configured to provide a force along the axis of the shaft 214 in response to compression. While the resilient member 218 in the illustrated embodiment is a coil spring, in other embodiments various other components can be used including, for example, bellows, foam, gas- or fluid-filled chambers, etc. In still other embodiments, other force-generating members can be used in place of the resilient member. For example, a known force can be provided via a magnetic force, an electromagnetic force, capacitive force, gravitational force, piezoelectric force (e.g., piezo-bender), pneumatic force, or other suitable approaches.

The contact member 210 includes a proximal flange 224, a distal flange 226, and a neck 228 connecting the proximal flange 224 and the distal flange 226. A channel 230 within the contact member 210 can be sized and configured to receive at least a portion of the shaft 214 of the sensor 208. The shape and dimensions of both the shaft 214 and the channel 230 can vary, for example they can each have circular, elliptical, rectangular, irregular, or other such cross-sectional shapes, so long as the channel 230 is dimensioned to receive at least a portion of the shaft 214. The proximal flange 224 of the contact member 210 includes a proximal contact face 232 with an opening defining the channel 230. When the shaft 214 is received within the channel 230, the distal end 222 of the resilient member 218 can engage the proximal contact face 232 of the proximal flange 224. The shaft 214 can be free to slide within the channel 230. The proximal flange 224 can have a stop contact face 234 opposite the proximal contact face 232. As described in more detail below, in some embodiments the stop contact face 234 of the proximal flange 224 can be configured to engage with a stop member so as to limit the movement of the contact member 210. The distal flange 226 of the contact member 210 can be configured to engage with a surface to be measured, for example a flexible membrane or interface of the catheter as described in more detail below. In particular, the distal contact face 236 can be configured to engage the surface to be measured.

FIGS. 2B and 2C are schematic views of a self-calibrating sensor assembly 206 engaged with a drainage catheter 102 in a sensing mode and a calibration mode, respectively. As shown in FIGS. 2B-2C, the drainage catheter 102 has a flexible interface member 238 in communication (e.g., physical contact) with the contact member 210. In operation, the flexible interface member 238 of the drainage catheter 102 expands or inflates as fluid pressure within the drainage catheter 102 increases (e.g., representing an increase in ICP), and retracts or deflates as fluid pressure within the drainage catheter 102 decreases (e.g., representing a decrease in ICP). The fluctuations of the flexible interface member 238 (e.g., representative of fluctuations in ICP) are communicated to the contact member 210. That is, when the pressure within the flexible interface member 238 increases, the flexible interface member 238 applies more pressure against the contact member 210. The pressure applied by the flexible interface member 238 against the contact member 210 can be detected by the sensor 208. For example, if the sensor 208 is a force sensor, the pressure can be calculated by dividing the detected force at sensor 208 by the surface area of the contact member 210 that is in contact with the flexible interface member 238.

The drainage catheter 102 can be made of polyurethane tubing and/or other suitable materials for sealing the bodily fluid therein. The flexible interface member 238 of the drainage catheter 102 can be a flexible membrane or diaphragm made from substantially flexible materials that are sensitive to changes in pressure and the application of small forces thereon, such as the forces applied when pressure changes within the drainage catheter 102. For example, the flexible interface member 238 can be made from ether- or ester-based materials. In other embodiments, the flexible interface member 238 can be made from other suitable flexible materials. The flexible interface member 238 can be attached to the drainage catheter 102 via molding, adhesives, and/or other suitable connection techniques, or the flexible interface member 238 can be integrally formed with the drainage catheter 102. For illustrative purposes, the flexible interface member 238 are shown protruding outwardly from the sides of the drainage catheter 102. However, under normal conditions when no external pressures are applied to the flexible interface member 238, the flexible interface member 238 can be in a relaxed or flaccid state such that the material of the flexible interface member 238 is not stretched or placed under tension. Accordingly, the flexible interface member 238 may appear substantially in line with the sidewall of the drainage catheter 102. Then, when a force acts on the flexible interface member 238, it can move inwardly or outwardly depending on the force applied. In other embodiments, the flexible interface member 238 may be configured such that the normal, relaxed state of the material causes the flexible interface member 238 to protrude outwardly or inwardly.

In various embodiments, the flexible interface member 238 of the drainage catheter 102 and the sensor assembly 206 can be contained within a housing 240. The housing 240 may be a durable case or container that provides protection for the flexible interface member 238, the sensor assembly 206, and/or any other system components (e.g., electronics) stored therein, and further include attachment features that position the flexible interface member 238 and the sensor assembly 206 appropriately with respect to each other. For example, the housing 240 can include protrusions or grooves (not visible) that receive the drainage catheter 102 and position the flexible interface member 238 to be in communication the contact member 210. In certain embodiments, the sensor assembly 206 can be pre-packaged within the housing 240 such that the contact member 210 is affixed in a desired position. The drainage catheter 102 can then be positioned within the housing 240 such that the flexible interface member 238 is in communication (e.g., physically in contact) with the contact member 210. For example, the housing 240 may include attachment features that appropriately position the flexible interface member 238 with respect to the contact member. This embodiment facilitates use of the housing 240 and the sensor assembly 206 with previously-implanted drainage catheters. In other embodiments, the housing 240 can be preassembled with the drainage catheter 102 and the sensor assembly 206 such that the flexible interface member 238 and the contact member 210 are affixed in the desired positions with the flexible interface member 238 contacting or attached to the sliding contact member. In further embodiments, the proximal elements of the drainage system 100 can be assembled within the housing 240 during or after the drain implantation procedure. In still further embodiments, the housing 240 can be omitted, and the proximal elements of the drainage system 100 can be positioned appropriately with respect to each other and with respect to the patient 101 using other suitable means. In some embodiments, the housing 240 can be disposed within the housing 128 of the valve device (FIGS. 1A-1B). In other embodiments, the sensor assembly 206 can be positioned within the housing 128 of the valve device (FIGS. 1A-1B) and the separate housing 240 can be omitted.

The sensor assembly 206 further includes an actuator 242 operably coupled to the sensor 208 and configured to move the sensor 208 with respect to the drainage catheter 102. For example, the actuator 242 can move the sensor 208 between a first position as shown in FIG. 2B (e.g., sensing mode) and a second position as shown in FIG. 2C (e.g., calibration mode). The actuator 242 can be, for example, piezoelectric, microelectromechanical, pneumatic, or can include other suitable actuator mechanism for translating the sensor 208 with respect to the drainage catheter 102.

Stop member 244 is disposed within the housing 240 and beneath the stop contact face 234 of the contact member 210. The stop member 244 can be, for example, forked piece of rigid material fixedly attached to the housing 240. The stop member 244 can take a number of other forms, for example an annulus surrounding a portion of the contact member 210, a single rigid component that engages the contact member 210 in only single location, or other such form suitable for limiting the distal movement of the contact member 210 with respect to the drainage catheter 102. The stop member 244 can be arranged such that it is spaced apart from the stop contact face 234 of the contact member 210 in sensing mode (FIG. 2B). In this position, the contact member 210 can move along the axis of the shaft 214 in response to changes in pressure within the drainage catheter 102 as communicated via the flexible interface member 238. For example, an increase in pressure in the drainage catheter 102 can cause the flexible interface member 238 to expand further outwardly, thereby exerting a proximal or upward force on the contact member 210. The contact member 210 can slide proximally with respect to the shaft 214. This proximal force can be communicated to the sensor 208 via the resilient member 218. For example, the proximal contact face 232 of the contact member 210 can engage the distal end 222 of the resilient member 218, while the proximal end 220 of the resilient member 218 can engage the collar 216 of the sensor 208. The resilient member 218 can have a selected stiffness or rigidity to communicate the proximal force exerted on the contact member 210 by the flexible interface member 238 to the sensor 208. Similarly, a decrease in pressure in the drainage catheter 102 can result in a partial deflation of the flexible interface member 238, thereby reducing the proximal or upward force exerted on the contact member 210. The contact member 210 may move distally resulting in a reduced proximal force communicated to the sensor 208 via the resilient member 218.

In the calibration mode (FIG. 2C), the sensor 208 can be advanced distally via the actuator 242, thereby compressing the resilient member 218. Compression of the resilient member 218 exerts a distal force on the contact member 210 until the stop contact face 234 of the contact member 210 engages (e.g., comes into physical contact with) the stop member 244.

In various embodiments, the sensor assembly 206 can be configured to measure negative pressures within the drainage catheter 102. When the flexible interface member 238 is subject to negative pressures, it may retract and, as a result, may come out of contact with the contact member 210. This loss of contact prevents the contact member 210 from translating the movement of the flexible interface member 238 to pressure or force measurements. Accordingly, the sensor assembly 206 can include features that maintain contact between the contact member 210 and the flexible interface member 238, regardless of the direction of movement of the flexible interface member 238. For example, when the drainage catheter 102 and the sensor assembly 206 are preassembled (e.g., within the housing 240), the flexible interface member 238 and the contact member 210 can be permanently bonded together.

The processing device 246 can be operably coupled to the sensor assembly 206 and/or other features of the drainage system 100 (e.g., valves). The processing device 246 can include or be part of a device that includes a hardware controller that interprets the signals received from input devices (e.g., the sensor 208, other sensors, user input devices, etc.) and communicates the information to the processing device 246 using a communication protocol. The processing device 246 may be a single processing unit or multiple processing units in a device or distributed across multiple devices. The processing device 246 may communicate with the hardware controller for devices, such as for a display that displays graphics and/or text (e.g., LCD display screens). The processing device 246 can also be in communication with a memory (e.g., within the housing 240) that includes one or more hardware devices for volatile and non-volatile storage, and may include both read-only and writable memory. For example, a memory may comprise random access memory (RAM), read-only memory (ROM), writable non-volatile memory, such as flash memory, hard drives, floppy disks, CDs, DVDs, magnetic storage devices, tape drives, device buffers, and so forth. A memory is not a propagating electrical signal divorced from underlying hardware, and is thus non-transitory. In certain embodiments, the processing device 246 can also be coupled to a communication device capable of communicating wirelessly or wire-based with a network node. The communication device may communicate with another device or a server through a network using, for example, TCP/IP protocols.

The processing device 246 can execute automated control algorithms to initiate, terminate, and/or adjust operation of one or more features of the sensor assembly 206 and/or receive control instructions from a user. The processing device 246 can further be configured to provide feedback to a user based on the data detected by the sensor assembly 206 via an evaluation/feedback algorithm. For example, the processing device 246 can be configured to provide clinicians, patients, and/or other users with a patient's pressure level at a site of excess body fluid (e.g., ICP), indicators of when a threshold pressure level is exceeded, and/or other pressure-related information based on the information received from the sensor 208. This information can be provided to the users via a display (e.g., a monitor on a computer, tablet computer, or smart phone; not shown) communicatively coupled to the processing device 246.

The processing device 246 can executed automated control algorithms to initiate a calibration process. For example, the processing device 246 can be operably coupled to the actuator 242 and the sensor 208. In operation, the processing device 246 can receive output from the sensor 208. In a sensing mode (FIG. 2B), the processing device 246 can cause the actuator 242 to position the sensor 208 with respect to the drainage catheter 102 such that pressure measurements can be obtained. The processing device 246 can periodically calibrate the sensor assembly 206 at predetermined intervals (e.g., weekly, monthly, annually, etc.). For example, the processing device 246 can cause the actuator 242 to advance the sensor 208 distally to a second position for a calibration mode (FIG. 2C). In calibration mode, the sensor 208 is moved to compress the resilient member 218 to a known tension, and then the known properties of the resilient member 218 are used to provide a reference force for calibration of the sensor 208. Additionally, the movement of the sensor 208 overcomes the pressure being measured (i.e., the pressure within the drainage catheter 102 as communicated via the flexible interface member 238), such that the system pressure does not influence the force applied to the sensor 208 in the calibration mode.

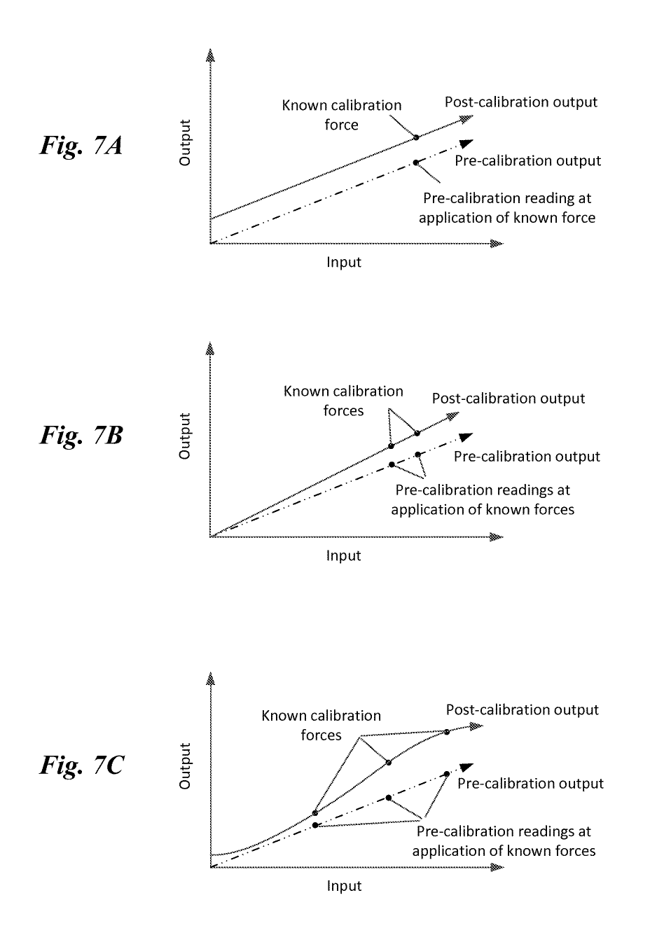

In the calibration mode, distal movement of the sensor 208 exerts a compressive force on the resilient member 218, which itself exerts a distal force on the contact member 210. The contact member 210 is urged distally until the stop contact face 234 of the contact member 210 engages the stop member 244. The contact member 210, resilient member 218, and stop member 244 can be configured so that in this position the internal pressure of the flexible interface member 238 is overcome. Since the force or pressure exerted by the contact member 210 on the flexible interface member 238 is sufficient to overcome the internal pressure of the flexible interface member 238, the sensor 208 in this position senses only the contribution from the resilient member 218. Furthermore, the stop member 244 ensures that the distance between the proximal contact face 232 of the contact member 210 and the collar 216 of the sensor 208 is fixed, and therefore the resilient member 218 is compressed a known amount. By using the known properties of the resilient member 218, the force detected by the sensor 208 in the calibration position can be determined. This determined force can provide a calibration point for use in re-calibrating the sensor 208 periodically. The resilient member 218 can be compressed to one known tension to allow a single-point correction (e.g., an offset) or compressed to multiple known tensions to allow multi-point calibration (e.g., correcting for an offset and change in sensor slope). These calibration calculations are described in more detail below with respect to FIGS. 7A-7C.

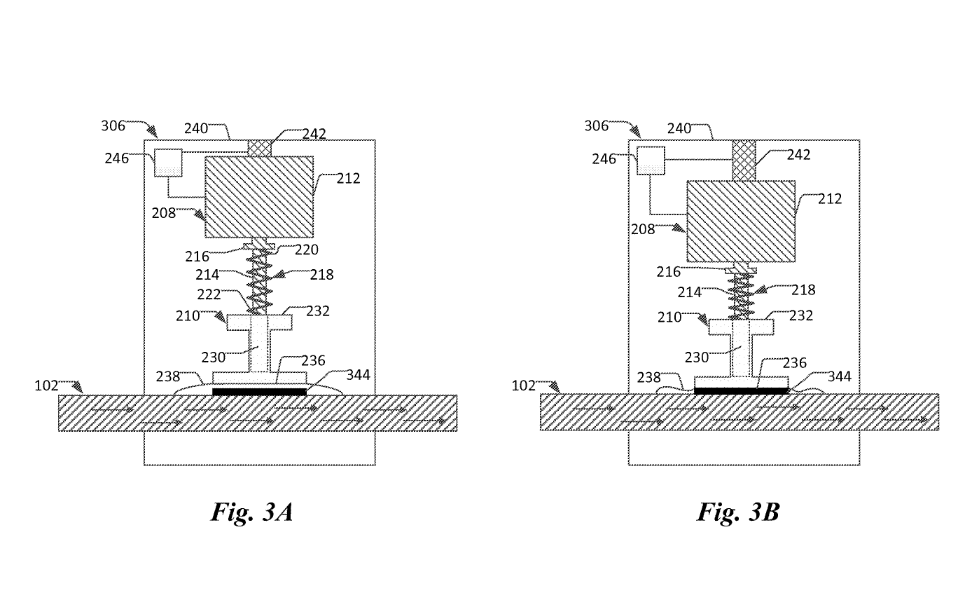

FIGS. 3A and 3B are schematic views of a self-calibrating sensor assembly 306 engaged with a drainage catheter 102 in a sensing mode and a calibration mode, respectively. Certain features of the sensor assembly 306 are at least generally similar to the sensor assembly 206 described above with respect to FIGS. 2A-2C. In the embodiment illustrated in FIGS. 3A and 3B, however, a stop member 344 is disposed within the flexible interface member 238 of the drainage catheter 102, and the flexible interface member 238 maintains fluid communication with the drainage catheter 102. The stop member 344, for example, can be a rigid component positioned within the flexible interface member 238 such that during the sensing mode (FIG. 3A), the contact member 210 slides along the shaft 214 in response to varying pressure of the flexible interface member 238. During sensing mode and under expected operating system pressure (e.g., ICP), the contact member 210 can have no contact with the stop member 344.

In the calibration mode, the processing device 246 can cause the actuator 242 to advance the sensor 208 distally (FIG. 3B). Distal movement of the sensor 208 exerts a compressive force on the resilient member 218, which itself exerts a distal force on the contact member 210. The contact member 210 is urged distally until the distal contact face 236 of the contact member 210 engages the stop member 344. The contact member 210, resilient member 218, and stop member 344 can be configured so that in this position the internal pressure of the flexible interface member 238 is overcome. Since the force or pressure exerted by the contact member 210 on the flexible interface member 238 is sufficient to overcome the internal pressure of the flexible interface member 238, the sensor 208 in this position senses only the contribution from the resilient member 218. Furthermore, the stop member 344 ensures that the distance between the contact member 210 and the collar 216 of the sensor 208 is fixed, and therefore the resilient member 218 is compressed a known amount. By using the known properties of the resilient member 218, the force detected by the sensor 208 in the calibration position can be determined. This determined force can provide a calibration point for use in re-calibrating the sensor 208 periodically.

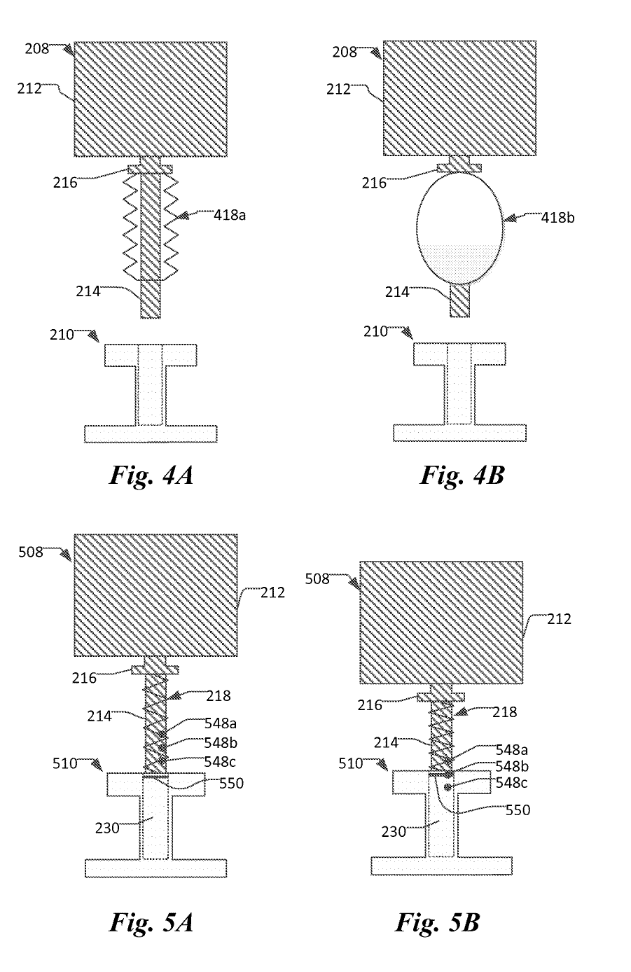

In the embodiments illustrated in FIGS. 2A-3B, the resilient member takes the form of a helical coil or spring. However, in various embodiments the resilient member can take a number of different forms. For example, as illustrated in FIG. 4A, the resilient member 418a is a bellows or other gas-filled chamber disposed about the shaft 214. The resilient member 418a is configured such that upon distal advancement of the sensor 208, the resilient member 418a is compressed between the collar 216 and the contact member 210. Based on the known spring rate of the bellows, the resilient member 418a can be used for calibration of the sensor 208 similar to the processes described above with respect to FIGS. 2A-3B. FIG. 4B illustrates another embodiment of a resilient member 418b. In this example, the resilient member 418b comprises a fluid-filled chamber. In some embodiments, the fluid-filled chamber of resilient member 418b can be configured such that, upon compression between the collar 216 and the contact member 210, the fluid flows through a resistant pathway to provide a known trainset response in pressure or force versus time. This can provide for a dynamic measurement with a time-dependent change in force or pressure, leading to a multi-point calibration of the sensor 208.

FIGS. 5A and 5B are schematic views of components of a self-calibrating sensor assembly utilizing electrical contacts shown in a sensing mode and a calibration mode, respectively. Certain features illustrated in FIGS. 5A and 5B can be at least generally similar to those described above with respect to FIGS. 2A-4B. For example, a sensor 508 includes a body 212, a shaft 214 projecting distally from the body 212, and a collar 216 connecting the body 212 and the shaft 214. However, the sensor 508 additionally includes a plurality of first electrical contacts 548a-c disposed on the shaft 214. In the illustrated embodiment there are three of the first electrical contacts 548a-c, however in other embodiments there may be fewer or greater contacts. The contact member 510 can likewise be similar to the contact member 210 described above with respect to FIGS. 2A-4B. However, the contact member 510 includes a second electrical contact 550 disposed within the channel 230. For example, the second electrical contact 550 can be a ring that spans the circumference of the channel 230. In sensing mode (FIG. 5A), there may be no contact between any of the first electrical contacts 548a-c and the second electrical contact 550.

In the calibration mode (FIG. 5B), the sensor 508 can be distally advanced, thereby compressing the resilient member 218 between the contact member 510 and the collar 216 of the sensor 508. In this position, the shaft 214 of the sensor 508 extends further into the channel 230 of the contact member 510. As a result, one or more of the first electrical contacts 548a-c may come into electrical contact with the second electrical contact 550. As illustrated in FIG. 5B, for example, first electrical contact 548b is in contact with second electrical contact 550. As the first electrical contacts 548a-c are disposed at different positions along the longitudinal axis of the shaft 214, electrical contact between each individual first electrical contact 548a-c and the second electrical contact 550 is indicative of the relative position of the contact member 510 and the sensor 508. This relative position also reflects a known compression of the resilient member 218. As a result, the resilient member 218 can exert a first known force on the sensor when the first electrical contact 548a is in electrical communication with the second electrical contact 550, thereby providing a first calibration point. Similarly, the resilient member 218 can exert a second known force on the sensor 208 when the first electrical contact 548b is in electrical communication with the second electrical contact 550, thereby providing a second calibration point, etc. The number of first electrical contacts 548a-c and/or the second electrical contact 550 can be varied, as can the configuration and orientation of each. For example, in some embodiments the first electrical contacts can be annular rings that surround the shaft 214 at different positions along the longitudinal length of the shaft 214. The first electrical contacts 548a-c and second electrical contact 550 can each be in electrical communication with a processing device (e.g., processing device 246 shown in FIGS. 2B-3B) which can control movement of the sensor 208 and can detect electrical communication between any of the first electrical contacts 548a-c and the second electrical contact 550. In some embodiments the multiple electrical contacts 548a-c can provide for a multi-point calibration. Additionally, in some embodiments the use of the multiple first electrical contacts 548a-c can eliminate the need to use a stop member. In some embodiments, electrical contacts 548a-c and 550 can be incorporated into the sensor assemblies 206 and 306 described above with respect to FIGS. 2B-3B.

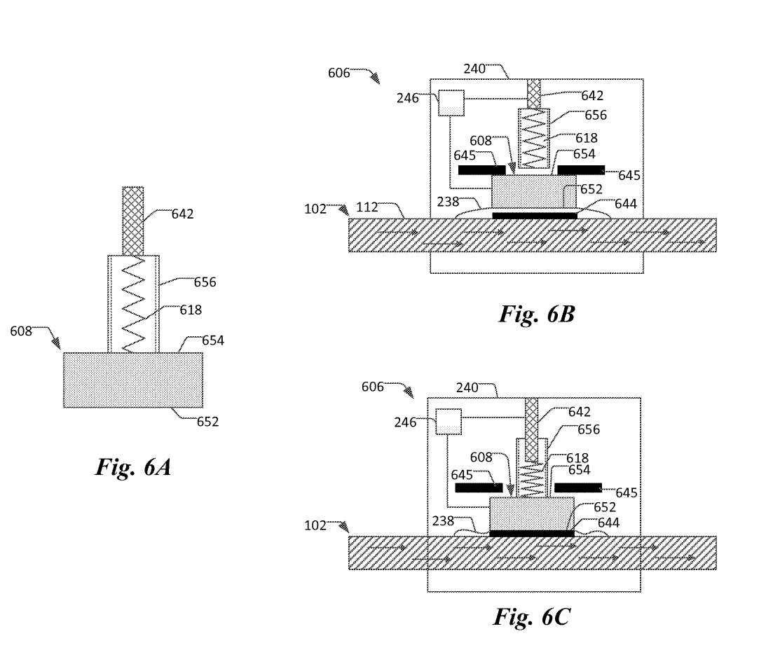

FIG. 6A is an exploded schematic view of components of a self-calibrating sensor assembly 606 (FIGS. 6B-6B) configured in accordance with yet another embodiment of the present technology. A sensor 608 has a distal sensing face 652 and an opposite proximal face 654. The sensor 608 can be, for example, a pressure sensor or a force sensor. A resilient member 618 is disposed adjacent the proximal face 654 of the sensor 608. The resilient member 618 can be, for example, a coil spring, or in other embodiments can be a fluid- or gas-filled chamber or other suitable component for exerting a force in response to compression. A guide member 656 is also disposed adjacent to the proximal face 654 of the sensor 608. The guide member 656 can be configured to retain the resilient member 618 in a desired orientation with respect to the sensor 608. For example, the guide member 656 can be a hollow column, opposing sidewalls, or other such structure configured to retain the resilient member 618 oriented so as to urge the sensor 608 distally in response to compression. An actuator 642 is disposed proximal to the resilient member 618 and is operably coupled to the resilient member 618 and the guide member 656 such that the actuator 642 can advance distally with respect to the resilient member 618, thereby compressing the resilient member 618 and urging the sensor 608 distally.

FIGS. 6B and 6C are schematic views of a self-calibrating sensor assembly 606 engaged with a drainage catheter 102 in a sensing mode and a calibration mode, respectively. Certain features of the sensor assembly 606 and the drainage catheter 102 can be at least generally similar to those described above with respect to FIGS. 2A-5B. As shown in FIGS. 6B and 6C, the drainage catheter 102 has a flexible interface member 238 in communication (e.g., physical contact) with the sensor 608. In operation, the fluctuations of the flexible interface member 238 (e.g., representative of fluctuations in ICP) are communicated to the sensor 608. In various embodiments, the flexible interface member 238 of the drainage catheter 102 and the sensor assembly 606 can be contained within the housing 240. The sensor assembly 606 further includes an actuator 642 which is operably coupled to the resilient member 618 and configured to move the resilient member 618 with respect to the drainage catheter 102. For example, the actuator 642 can move the resilient member 618 between a first position as shown in FIG. 6B (e.g., sensing mode) and a second position as shown in FIG. 6C (e.g., calibration mode). The actuator 642 can be, for example, piezoelectric, microelectromechanical, pneumatic, or other suitable actuator mechanism for translating the resilient member 618 with respect to the drainage catheter 102.

Stop member 644 is disposed within the flexible interface member 238 of the catheter 102. The stop member 644, for example, can be a rigid component positioned within the flexible interface member 238 such that during the sensing mode (FIG. 6A), the sensor 608 has no contact with the stop member 644. In other embodiments the stop member 644 can be disposed above the flexible interface member and configured to engage with a portion of the sensor 608 in calibration, similar to the arrangement described above with respect to FIGS. 2B and 2C. In still further embodiments, the stop member 644 can be disposed adjacent the external surface 112 of the catheter 102 on a side opposite the flexible interface member 238.

A second stop member 645 can be disposed adjacent to the proximal face 654 of the sensor 608. The second stop member 645 can retain the sensor 608 in position against the flexible interface member 238. In the sensing mode (FIG. 6B), the resilient member 618 does not participate in the force or pressure measured by the sensor 608. The sensor 608 is held in place with respect to the flexible interface member 238 via the second stop member 645 such that the only force or pressure measured is due to the flexible interface member 238 pushing (or pulling) relative to the second stop member 645 disposed adjacent the proximal face 654 of the sensor 608. In other embodiments, the resilient member 618 can be coupled to the sensor 608 and configured to generate a negative force when in the sensing mode (FIG. 6B), thereby pulling the sensor 608 against the second stop member 645. The negative force exerted by the resilient member 618 can be configured to be greater than any negative pressure to be measured within the drainage catheter 102. In some embodiments, only positive pressures are measured and no additional force is needed to retain the sensor 608 against the second stop member 645.

In the calibration mode (FIG. 6C), the actuator 242 advances the resilient member 618 thereby compressing the resilient member 618 against the sensor 608. Compression of the resilient member 618 urges the sensor 608 distally until the force overcomes the internal system pressure within the flexible interface member 238 of the drainage catheter 102. The resilient member 618 can exert a distal force on the sensor 608 until the sensing face 652 of the sensor 608 engages the stop member 644 within the flexible interface member 238.

The processing device 246 can be operably coupled to the sensor assembly 606 and/or other features of the drainage system 100 (e.g., valves). The processing device 246 can execute automated control algorithms to initiate a calibration process. For example, the processing device 646 can be operably coupled to the actuator 642 and the sensor 608. In operation, the processing device 646 can receive output from the sensor 608. In a sensing mode (FIG. 6B), the processing device 646 can cause the actuator 642 to position the sensor 608 with respect to the drainage catheter 602 such that pressure measurements can be obtained. The processing device 646 can then cause the actuator 642 to advance the sensor 608 distally to a second position for a calibration mode (FIG. 6C). In calibration mode, the resilient member 618 is moved distally until it urges the sensor 608 against the stop member 644. The known properties of the resilient member 618 are then used to provide a reference force for calibration of the sensor 608. Additionally, the movement of the sensor 608 overcomes the pressure being measured (i.e., the pressure within the drainage catheter 102 as communicated via the flexible interface member 638), such that the system pressure does not influence the force applied to the sensor 608 in the calibration mode.

### Selected Embodiments of Sensor Calibration Processes

FIG. 7A is a graph showing an example of a single-point calibration. The pre-calibration output line indicates the sensor output with respect to the pressure input prior to a calibration procedure. Calibration can be carried out as described above with respect to FIGS. 2A-6C. For example, in some embodiments a sensor can be advanced against a resilient member such as a spring until the resilient member is compressed to a known tension. The sensor output can be taken at this point of known tension of the resilient member. This output is indicated in FIG. 7A as the pre-calibration reading at application of known force. By comparing this point with the known calibration force, a new sensor calibration can be determined. For example, if the application of the known force provides an output that is 1 gram lower than the known force from the resilient member at the predetermined compression, the post-calibration output can be created as the pre-calibration output with 1 gram added (e.g., the post-calibration output can be generated as a simple offset of the pre-calibration output). If the sensor drift is reflected as a change in offset with the slope remaining relatively constant, then single-point calibration may be sufficient for some applications. This calibration approach can be applied equally to pressure and force sensors.

FIG. 7B is a graph showing an example of a two-point calibration. Similar to the process described above with reference to FIG. 7A, a pre-calibration output can be taken from a sensor under application of two known forces. For example, a sensor can be advanced to compress a resilient member to two separate, known tensions. The output at each of these points reflects the pre-calibration readings at application of known forces. By comparing these outputs to the known forces corresponding to the readings, a new post-calibration output can be determined. Since two points are used, the post-calibration output can be a new linear calibration, and need not be limited to a simple offset of the pre-calibration output.

FIG. 7C is a graph showing an example of a three-point calibration. This approach is similar to that of FIG. 7B, except that pre-calibration readings are taken at application of three separate known forces. for example, the sensor can be advanced to compress the resilient member to three separate, known tensions. The output at these points reflects the pre-calibration readings at application of known forces. These three points can then be used to create a new arbitrary calibration (e.g., non-linear) adapted to reflect the known force measurements. This multi-point approach can be expanded to the use of four, five, or more known calibration forces to more accurately determine an appropriate calibration, whether linear or nonlinear.

### EXAMPLES

1. A system, comprising:

a drainage catheter having an inlet and a flexible interface member positioned distally with respect to the inlet, wherein the inlet is configured to be in fluid communication with a site of excess body fluid within a human patient; and

a sensor assembly engaged with the flexible interface member and configured to measure the pressure and/or force at the flexible interface member, the sensor assembly comprising:
a sensor having a body and a shaft extending from the body;
a contact member slidably mated with the shaft, the contact member coupled to the flexible interface member;
a resilient member coupled to the sensor shaft and disposed between the contact member and the body; and
an actuator configured to move the sensor between a first position and a second position with respect to the drainage catheter,
wherein, in the first position, the sensor is positioned to measure the pressure and/or force at the flexible interface member, and
wherein, in the second position, the resilient member exerts a known force on the sensor.

2. The system of example 1 wherein the sensor assembly further comprises a stop member configured to engage the contact member when the sensor is in the second position.

3. The system of example 2 wherein the stop member is disposed between the flexible interface member and the resilient member.

4. The system of example 2 wherein the stop member is disposed within the flexible interface member.

5. The system of example 2 wherein the stop member is disposed adjacent the drainage catheter opposite the flexible interface member.

6. The system of any one of examples 1-5 wherein the resilient member comprises at least one of: a spring, a bellows, and a fluid-filled chamber.

7. The system of any one of examples 1-6 wherein in the second position the force exerted by the resilient member is sufficient to overcome the pressure at the flexible interface member.

8. The system of any one of examples 1-7 wherein the actuator is a first actuator, and wherein the system further comprises:

a valve device having a second actuator over an exterior surface of the drainage catheter, the second actuator being movable between an open position that allows body fluid flow through the drainage catheter, a closed position that at least substantially obstructs the body fluid flow through the drainage catheter, and intermediate positions that partially obstruct the body fluid flow through the catheter; and
a controller operatively coupled to the valve device and the sensor assembly, wherein the controller is configured to control the position of the second actuator in response to a predetermined condition of the sensor assembly.

9. The system of any one of examples 1-8 wherein the sensor assembly is biocompatible and MRI-safe.

10. The system of any one of examples 1-9 wherein the actuator is piezoelectric.

11. A system, comprising:

a catheter having an inlet configured to be in fluid communication with a site of excess body fluid within a patient and a flexible interface member spaced along the catheter apart from the inlet;

a sensor operably coupled to the flexible interface member and configured to detect pressure and/or force in the catheter via displacement of the flexible interface member;

an actuator operably coupled to the sensor and configured to move the sensor along a first axis with respect to the flexible interface member; and

a resilient member coupled to the sensor and configured to exert a force on the sensor in response to compression along the first axis.

12. The system of example 11 wherein the actuator is configured to move the sensor to a predefined position in which the resilient member exerts a known force upon the sensor.

13. The system of any one of examples 11-12 wherein the sensor is a pressure sensor, and wherein the resilient member is disposed between the pressure sensor and the actuator.

14. The system of any one of examples 11-12 wherein the sensor is a force sensor and comprises a contact member that engages the flexible interface member, wherein the system further comprises a stop member configured to engage the contact member when the sensor is moved by the actuator to a predetermined position.

15. The system of any one of examples 11-14 wherein the resilient member comprises at least one of: a spring, a bellows, and a fluid-filled chamber.

16. The system of any one of examples 11-15 wherein the actuator is a first actuator, the system further comprising:

a valve device having a second actuator over the catheter, wherein the second actuator is configured to apply incremental force to an exterior surface of the catheter to regulate body fluid flow through the catheter; and

a controller operatively coupled to the valve device and the sensor, the controller being configured to change the force applied to the catheter by the second actuator in response to a predetermined condition of the sensor.

17. A self-calibrating sensor assembly, comprising:

a sensor having a body and a shaft extending from the body along a first axis;

a contact member slidably mated with the shaft;

a resilient member coupled to the sensor shaft and disposed between the contact member and the body, the resilient member configured to exert a force upon the sensor in response to compression along the first axis; and

an actuator configured to move the sensor along the first axis.

18. The self-calibrating sensor assembly of example 17 wherein the contact member comprises a distal contact face configured to engage with a surface to be measured and a proximal contact face configured to engage with the resilient member, and wherein sliding of the contact member towards the sensor body compresses the resilient member.

19. The self-calibrating sensor assembly of any one of examples 17-18 wherein the contact member comprises a first electrical contact, and wherein the shaft comprises a second electrical contact, wherein connection between the first electrical contact and the second electrical contact indicates a first relative position between the contact member and the shaft.

20. The self-calibrating sensor assembly of example 19 wherein the shaft comprises a third electrical contact, wherein connection between the first electrical contact and the third electrical contact indicates a second relative position between the contact member and the shaft.

21. The self-calibrating sensor assembly of any one of examples 17-20, further comprising a processing device configured to receive an output of the sensor and to control the actuator.

22. The self-calibrating sensor assembly of example 21 wherein the processing device is further configured to:

initiate movement of the sensor, via the actuator, along the first axis to a first position such that the resilient member exerts a known force upon the sensor;

collect an output of the sensor at the first position; and

based on the collected output of the sensor at the first position, determine a calibration for the sensor.

23. The self-calibrating sensor assembly of any one of examples 17-22 wherein the resilient member comprises at least one of: a spring, a bellows, and a fluid-filled chamber.

24. A self-calibrating sensor assembly, comprising:

a pressure sensor having a sensing face that faces a first direction;

a resilient member having a first end coupled to the pressure sensor and a second end opposite the first end, the resilient member configured to exert a force upon the pressure sensor along the first direction in response to compression; and

an actuator operably coupled to the second end of the resilient member, the actuator configured to advance the second end of the resilient member in the first direction, thereby compressing the resilient member.

25. The self-calibrating sensor assembly of example 24 further comprising a guide configured to receive the resilient member and retain its orientation with respect to the pressure sensor.

26. The self-calibrating sensor assembly of example 25 wherein the guide comprises a hollow column configured to at least partially surround the resilient member.

27. The self-calibrating sensor assembly of any one of examples 24-26 further comprising a processing device configured to receive an output of the pressure sensor and to control the actuator.

28. The self-calibrating sensor assembly of example 27 wherein the processing device is further configured to:

advance the second end of the resilient member, via the actuator, to a first position such that the resilient member exerts a known force upon the sensor;

collect an output of the pressure sensor at the first position; and

based on the collected output of the pressure sensor at the first position, determine a calibration for the pressure sensor.

29. The self-calibrating sensor assembly of any one of examples 24-28 wherein the actuator is piezoelectric.

30. A method for calibrating a sensor assembly for detecting a pressure at a location within a catheter, the method comprising:

disposing a sensor assembly adjacent the catheter, the sensor assembly comprising—
a sensor coupled to a contact member in contact with the catheter; and
a resilient member disposed adjacent the sensor, the resilient member configured to exert a force upon the sensor in response to compression along a first axis;

advancing the sensor along the first axis towards the catheter to a first position, thereby compressing the resilient member; and

collecting a sensor output at the first position.

31. The method of example 30 wherein a stop member engages the contact member at the first position.

32. The method of any one of examples 30-31 wherein the resilient member exerts a known force upon the sensor at the first position.

33. The method of example 32 further comprising determining a calibration based on the sensor output at the first position.

34. The method of example 33 wherein the sensor output at the first position is not indicative of the pressure at the location within the catheter.

35. The method of any one of examples 33-34 further comprising:

after determining the calibration, retracting the sensor along the first axis away from the first position and to a second position, thereby decreasing compression of the resilient member; and

collecting a sensor output at the second position.

36. The method of example 35, wherein the sensor output at the second position is indicative of the pressure at the location within the catheter.

### CONCLUSION

From the foregoing, it will be appreciated that specific embodiments of the present technology have been described herein for purposes of illustration, but that various modifications may be made without deviating from the spirit and scope of the disclosure. For example, the resilient members illustrated in FIGS. 4A and 4B can be included in the sensor assemblies 206, 306, and/or 606 of FIGS. 2A-C, 3A-B, and 6A-C, respectively. Similarly, the electrical contacts 548a-c and 550 of FIGS. 5A and 5B can be included in any of the sensor assemblies 206, 306, and/or 606 of FIGS. 2A-C, 3A-B, and 6A-C, respectively. Aspects of the disclosure described in the context of particular embodiments may be combined or eliminated in other embodiments. Further, while advantages associated with certain embodiments of the disclosure have been described in the context of those embodiments, other embodiments may also exhibit such advantages, and not all embodiments need necessarily exhibit such advantages to fall within the scope of the disclosure. Accordingly, embodiments of the disclosure are not limited except as by the appended claims.

1-36. (canceled)

37. A self-calibrating sensor system configured to measure a pressure and/or force at a flexible interface member subject to fluid pressure, the sensor assembly comprising:
a sensor having a body and a shaft extending from the body;
a contact member slidably mated with the shaft, the contact member coupled to the flexible interface member;
a force-generating member coupled to the sensor shaft and disposed between the contact member and the body; and
an actuator configured to move the sensor between a first position and a second position with respect to the flexible interface member,
wherein, in the first position, the sensor is positioned to measure the pressure and/or force at the flexible interface member, and in the second position, the force-generating member exerts a known force on the sensor.

38. The system of claim 37, wherein the sensor assembly further comprises a stop member configured to engage the contact member when the sensor is in the second position.

39. The system of claim 37, wherein the force-generating member comprises a resilient member selected from the group consisting of: a spring, a bellows, a foam-filled chamber, a gas-filled chamber, and a fluid-filled chamber.

40. The system of claim 37, wherein the force-generating member comprises a member capable of generating a known force using at least one of the following: a magnetic force, an electromagnetic force, a capacitive force, a gravitational force, a piezoelectric force, and a pneumatic force.

41. The system of claim 37, wherein in the second position the force exerted by the force-generating member is sufficient to overcome pressure at the flexible interface member.

42. The system of claim 37, wherein the actuator is a first actuator, wherein the flexible interface member is provided in association with a tube, and wherein the system further comprises:
a valve device having a second actuator, the second actuator being movable between an open position that allows fluid to flow through the tube and a closed position that at least substantially obstructs fluid from flowing through the tube; and
a controller operatively coupled to the valve device and the sensor assembly, wherein the controller is configured to control the position of the second actuator in response to a predetermined condition of the sensor assembly.

43. The system of claim 37, wherein the flexible interface member is provided in association with a catheter having an inlet configured to be in fluid communication with a site of body fluid.

44. The system of claim 37, wherein the actuator is piezoelectric.

45. The system of claim 37, wherein the contact member comprises a distal contact face configured to engage with a surface of the flexible interface member and a proximal contact face configured to engage with the force-generating member, and wherein sliding of the contact member towards the sensor body compresses the force-generating member.

46. The system of claim 37, wherein the contact member comprises a first electrical contact, and wherein the shaft comprises a second electrical contact, wherein connection between the first electrical contact and the second electrical contact indicates a first relative position between the contact member and the shaft.

47. The system of claim 46, wherein the shaft comprises a third electrical contact, wherein connection between the first electrical contact and the third electrical contact indicates a second relative position between the contact member and the shaft.

48. The system of claim 37, further comprising a processing device configured to receive an output of the sensor and to control the actuator.

49. The self-calibrating sensor assembly of claim 48, wherein the processing device is further configured to:
initiate movement of the sensor, via the actuator, to the first position such that the force- generating member exerts a known force upon the sensor;
collect an output of the sensor at the first position; and
based on the collected output of the sensor at the first position, determine a calibration for the sensor.

50. A sensor assembly configured to measure a pressure and/or force at a flexible interface member subject to fluid pressure, the sensor assembly comprising:
a sensor operably coupled to the flexible interface member and configured to detect fluid pressure and/or force via displacement of the flexible interface member;
an actuator operably coupled to the sensor and configured to move the sensor along a first axis with respect to the flexible interface member; and
a force-generating member coupled to the sensor and configured to exert a force on the sensor in response to compression along the first axis.

51. The sensor assembly of claim 50, wherein the actuator is configured to move the sensor to a predefined position in which the force-generating member exerts a known force upon the sensor.

52. The sensor assembly of claim 50, wherein the sensor is a force sensor and comprises a contact member that engages the flexible interface member, wherein the system further comprises a stop member configured to engage the contact member when the sensor is moved by the actuator to a predetermined position.

53. The sensor assembly of claim 50, wherein the force-generating member comprises a resilient member selected from the group consisting of: a spring, a bellows, a foam-filled chamber, a gas-filled chamber, and a fluid-filled chamber.

54. The sensor assembly of claim 50, wherein the force-generating member comprises a member capable of generating a known force using at least one of the following: a magnetic force, an electromagnetic force, a capacitive force, a gravitational force, a piezoelectric force, and a pneumatic force.

55. The sensor assembly of claim 50, wherein the actuator is a first actuator, wherein the flexible interface member is provided in association with a tube, and wherein the system further comprises:
a valve device having a second actuator, wherein the second actuator is configured to regulate fluid flow through the tube; and
a controller operatively coupled to the valve device and the sensor, the controller being configured to control the second actuator in response to a predetermined condition of the sensor.

56. A self-calibrating sensor assembly, comprising:
a pressure sensor having a sensing face that faces a first direction;
a resilient member having a first end coupled to the pressure sensor and a second end opposite the first end, the resilient member configured to exert a force upon the pressure sensor along the first direction in response to compression; and
an actuator operably coupled to the second end of the resilient member, the actuator configured to advance the second end of the resilient member in the first direction, thereby compressing the resilient member.

57. The self-calibrating sensor assembly of claim 56, further comprising a guide configured to receive the resilient member and retain its orientation with respect to the pressure sensor.

58. The self-calibrating sensor assembly of claim 57, wherein the guide comprises a column configured to at least partially surround the resilient member.

59. The self-calibrating sensor assembly of claim 56, further comprising a processing device configured to receive an output of the pressure sensor and to control the actuator.

60. The self-calibrating sensor assembly of claim 59, wherein the processing device is further configured to:
advance the second end of the resilient member, via the actuator, to a first position such that the resilient member exerts a known force upon the sensor;
collect an output of the pressure sensor at the first position; and
based on the collected output of the pressure sensor at the first position, determine a calibration for the pressure sensor.

61. A method for calibrating a sensor assembly for detecting a pressure at a location within a structure including a lumen through which fluids can flow, the method comprising:
disposing a sensor assembly adjacent the structure, the sensor assembly comprising a sensor coupled to a contact member in contact with the structure and a resilient member disposed adjacent the sensor, the resilient member configured to exert a force upon the sensor in response to compression along a first axis;
advancing the sensor along the first axis towards the structure to a first position, thereby exerting force against the resilient member;
collecting a sensor output at the first position; and
determining a calibration based on the sensor output at the first position.

62. The method of claim 61, wherein a stop member engages the contact member at the first position.

63. The method of claim 61, wherein the resilient member exerts a known force upon the sensor at the first position.

64. The method of claim 61, further comprising:
after determining the calibration, retracting the sensor along the first axis away from the first position and to a second position, thereby decreasing compression of the resilient member; and
collecting a sensor output at the second position.
